(19) United States
(12) Patent Application Publication
(10) Pub. No.: US 2025/0292874 A1
(43) Pub. Date: Sep. 18, 2025

(54) DEVICE AND METHOD FOR TRAINING DATA GENERATION THROUGH OBJECT DIVERSIFICATION OF STRUCTURAL FORMULA

(71) Applicant: LG MANAGEMENT DEVELOPMENT INSTITUTE CO., LTD., Seoul (KR)

(72) Inventors: Ahra Jo, Seoul (KR); Yeon Sik JO, Seoul (KR); Chang Young PARK, Seoul (KR); Soon Young LEE, Seoul (KR)

(21) Appl. No.: 19/211,656

(22) Filed: May 19, 2025

Related U.S. Application Data
(63) Continuation of application No. PCT/KR2023/018694, filed on Nov. 20, 2023.

(30) Foreign Application Priority Data
Nov. 18, 2022 (KR) 10-2022-0155035

(51) Int. Cl. G16C 20/30; G06N 20/00; G16C 20/70

(52) U.S. Cl. CPC G16C 20/30; G06N 20/00; G16C 20/70

(57) ABSTRACT

A device and method for training data generation through object diversification of a chemical structure are disclosed. The device comprises a processor and at least one memory electrically connected to the processor. The at least one memory stores one or more feature variables determining the features of objects expressing structural formulas; and a first setpoint set determined in advance for each of the one or more feature variables. The method comprises loading first chemical formula data; setting setpoint(s) of the one or more feature variables as the first setpoint set; generating a first structural formula on the basis of the first chemical formula by applying an object set to the first setpoint set; acquiring, in response to an input changing the setpoint(s) of one or more feature variables, a second setpoint set; generating a second structural formula by applying an object set to the second setpoint set; and generating the training data including the generated second structural formula.

### CROSS REFERENCE TO RELATED APPLICATION

This application is a Bypass Continuation of International Patent Application No. PCT/KR2023/018694, filed on Nov. 20, 2023, which claims priority from and the benefit of Korean Patent Application No. 10-2022-0155035, filed on Nov. 18, 2022, which is hereby incorporated by reference for all purposes as if fully set forth herein.

### BACKGROUND

### Field of the Invention

Embodiments of the invention relate generally to a device and a method for training data generation through object diversification of a chemical structural formula, and, more particularly, the present invention relates to a device and a method for training data generation, which device and method are capable of mass-producing a machine-readable representation of a chemical structural formula by diversifying setpoints of objects constituting the chemical structural formula based on chemical formula data including information about one or more node(s) and information about one or more edge(s).

### Discussion of the Background

In developing an artificial intelligence model that recognizes a two dimensional written chemical structural formula, generating training data using only a conventional chemical structural formula representation generation program does not reflect the diversity of actual structural formula data, and thus it is difficult to expect high performance of the artificial intelligence model.

In a conventional chemical structural formula representation generation program, the structural formula can be generated only in a fixed format, and it is difficult to take into account varying thicknesses, colors, or intervals of line segments representing bonds of the chemical structural formula, fonts, sizes, colors, or the like of characters representing atoms.

In addition, in order to improve the performance of the artificial intelligence model, a large amount of training data is required, and since the conventional chemical formula representation generation program can generate only one image at a time, it takes a lot of time to process data to generate additional data.

Conventional programs also suffer from the difficulty that, in training an artificial intelligence model using a vision technology, coordinate information and class information of atoms and bonding regions need to be derived from the structural formula training data, but such information cannot be acquired by the conventional chemical structural formula representation generation program. Acquiring coordinate information and class information of an atom and a bonding region by hand is difficult, so there is a need in the art for a chemical structural formula recognition program that can do this in an automated way.

The above information disclosed in this Background section is only for understanding of the background of the inventive concepts, and, therefore, it may contain information that does not constitute prior art.

### SUMMARY

The present invention provides a device and a method for generating training data, which device or method may mass-produce various formats of training data in order to train an artificial intelligence model which recognizes a chemical structural formula in an improved and more useful way.

The objects to be achieved by the present invention are not limited to the aforementioned objects, and other objects, which are not mentioned above, will be apparent to a person having ordinary skill in the art from the following description.

Additional features of the inventive concepts will be set forth in the description which follows, and in part will be apparent from the description, or may be learned by practice of the inventive concepts.

According to an aspect of the present invention, a device for training data generation can comprise a processor and at least one memory electrically connected to the processor, wherein the at least one memory can store at least one feature variable for determining a feature of an object representing a structural formula, and a first setpoint set predetermined for each of the at least one feature variable(s), and wherein the processor can load first chemical formula data including information about at least one node and information about at least one edge, and the at least one feature variable, can set a setpoint of each of the at least one feature variable(s) to the corresponding first setpoint set, can generate, based on the first chemical formula data, a first structural formula by using an object set that is applied to the first setpoint set for each of the at least one feature variable(s), can acquire, according to an input of changing the setpoint of the at least one feature variable(s), a second setpoint set in which the setpoint of each corresponding feature variable is changed, can generate a second structural formula using an object set that is applied to the second setpoint set, and can generate training data including the generated second structural formula.

In some embodiments of the inventive device, a new object constituting a node of the second structural formula can be formed by being annotated with the setpoint set for the feature variable of the corresponding existing object.

In some embodiments of the inventive device, a new object constituting an edge of the second structural formula can be formed by being annotated with the setpoint set for the feature variable of the corresponding existing object.

In some embodiments of the inventive device, a new object constituting a bracket of the second structural formula can be formed by being annotated with the setpoint set for the feature variable of the corresponding existing object.

In some embodiments of the inventive device, an object constituting the bracket and the object constituting the edge in the second structural formula can be formed by annotating a point where the object constituting the bracket and the object constituting the edge intersect with each other.

In some embodiments of the inventive device, the training data generation device can include a display outputting a structural formula, the processor can acquire a second setpoint set at each of a plurality of time points, and the display can output the first structural formula and the second structural formula and can output the second structural formula separately for each time point at which the changed second setpoint set is acquired.

In some embodiments of the inventive device, each of the at least one feature variable(s) can include at least one of a type, a thickness, a length, a size, and a color of the object, a spacing between objects, and an angle at which the object is tilted.

In some embodiments of the inventive device, the training data can be used for training an artificial intelligence model for predicting chemical formula data including information about at least one node and information about at least one edge.

According to another aspect, the present invention can include a method for training data generation, which method can be performed by a training data generation device and can comprise storing, by the training data generation device, at least one feature variable for determining a feature of an object representing a structural formula, and a first setpoint set predetermined for each of the at least one feature variable(s), loading first chemical formula data including information about at least one node and information about at least one edge, and the at least one feature variable(s), setting a setpoint of each of the at least one feature variable(s) to the respective first setpoint set, generating, based on the first chemical formula data, a first structural formula by using a first object set and applying the first object set to the first setpoint set for each feature variable, acquiring for each at least one feature variable, according to an input of changing the setpoint(s) of the at least one feature variable(s), a second setpoint set in which the setpoint of the corresponding feature variable is changed relative to the first setpoint set, generating a second structural formula using a second object set that is applied to the second setpoint set, and generating training data including the generated second structural formula. In some embodiments, the first object set can be identical with the second object set, and, in other embodiments, the first object set can be different from the second object set.

In some embodiments of the inventive method, an object constituting a node of the second structural formula can be formed by being annotated with the second setpoint set(s) for the feature variable(s) of the corresponding object of the second object set.

In some embodiments of the inventive method, an object constituting an edge of the second structural formula can be formed by being annotated with the second setpoint set(s) for the feature variable(s) of the corresponding object of the second object set.

In some embodiments of the inventive method, an object constituting a bracket of the second structural formula can be formed by being annotated with the second setpoint set(s) for the feature variable(s) of the corresponding object of the second object set.

In some embodiments of the inventive method, an object constituting a bracket and an object constituting an edge in the second structural formula can be formed by annotating a point where the object constituting the bracket and the object constituting the edge intersect with each other.

In some embodiments of the inventive method, the at least one feature variable can include at least one of a type, a thickness, a length a size, and a color of the object, a spacing between objects, and an angle at which the object is tilted.

In some embodiments, the inventive method can include a computer program stored in a computer-readable recording medium, coupled with a device that is hardware, to execute the method for generating training data according to the method of claim 9.

According to the present invention, various types of structural formula training data that are not in fixed formats can be generated. Further, a large amount of structural formula training data can be generated from the data of one structural formula.

In addition, a setpoint may be assigned to the structural formula generated by the training data generation device of the present invention for each feature variable of an object constituted by the chemical structural formula being evaluated. This facilitates annotation with a set value assigned to each object, can reduce an error occurrence rate of an annotation task, and can also reduce a cost in a step of verifying whether an annotation task result is correctly performed.

As is detailed in the following description, the performance of an artificial intelligence model that recognizes a chemical structural formula can be significantly improved by utilizing the structural formula generated by the training data generation device of the present invention.

The effects of the present invention are not limited to the aforementioned effect, and other effects, which are not mentioned above, will be apparent to a person having ordinary skill in the art from the following disclosure.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a block diagram of a device for training data generation according to an embodiment of the present invention.

FIG. 2 is a diagram for describing a process of generating a structural formula based on chemical formula data according to an embodiment of the present invention.

FIG. 3A illustrates a first structural formula generated by a predetermined setpoint, and FIG. 3B illustrates a second structural formula in which setpoints of some characters, numbers, and symbols are changed according to an embodiment of the present invention.

FIG. 4A illustrates a first structural formula generated by a predetermined setpoint, and FIG. 4B illustrates a second structural formula in which setpoints of some nodes and edges (atoms and bonds between atoms) are changed according to an embodiment of the present invention.

FIG. 5A illustrates a first structural formula generated by a predetermined setpoint, and FIG. 5B illustrates a second structural formula in which setpoints of some nodes and edges are changed according to an embodiment of the present invention.

FIG. 6A illustrates a first structural formula generated by a predetermined setpoint, and FIG. 6B illustrates a second structural formula in which setpoints of some nodes and edges are changed according to an embodiment of the present invention.

FIG. 7A illustrates a first structural formula generated by a predetermined setpoint, and FIG. 7B illustrates a second structural formula in which setpoints of some nodes and edges are changed according to an embodiment of the present invention.

FIG. 8A illustrates a first structural formula generated by a predetermined setpoint, and FIG. 8B illustrates a second structural formula in which setpoints of some features, atomic electrical charges, are changed according to an embodiment of the present invention.

FIG. 9A illustrates a first structural formula generated by a predetermined setpoint, and FIGS. 9B and 9C illustrate a second structural formula in which setpoints of some nodes and edges are changed according to an embodiment of the present invention.

FIG. 10 illustrates a structural formula in which objects constituting nodes are annotated according to an embodiment of the present invention.

FIG. 11 illustrates a structural formula in which objects constituting edges are annotated according to an embodiment of the present invention.

FIG. 12A-12I illustrate various types of objects constituting edges of chemical structural formulas according to an embodiments of the present invention.

FIG. 13A-13C illustrate a structural formula in which objects constituting brackets are annotated according to an embodiment of the present invention.

FIG. 14 illustrates a structural formula in which a point where a bracket and an object intersect is annotated according to an embodiment of the present invention.

FIG. 15 is a block diagram for describing a method for generating training data according to an embodiment of the present invention.

### DETAILED DESCRIPTION

The present inventors have unexpectedly found new ways to use artificial intelligence (AI) to encode chemical structural formulas in machine-readable formats. Improved machine recognition of chemical structural formulas will have many useful applications in science and industry. In the new world of AI, traditional studies of organic structure/property relationships can be taken to whole new realms of efficiency and completeness. Efforts to find the right organic materials for very specific applications will be greatly enhanced. AI methods can be expected to improve syntheses and thus reduce costs in many industries. Additionally, prediction of unknown properties of compounds having known structures will be made much easier, with many beneficial consequences.

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one element's relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Advantages and features of the present invention, and methods for accomplishing the same will be more clearly understood from embodiments described in detail below with reference to the accompanying drawings. However, the present invention is not limited to the embodiments set forth below, and may be embodied in various different forms. The present embodiments are just for rendering the disclosure of the present invention complete and are set forth to provide a complete understanding of the scope of the invention to a person with ordinary skill in the technical field to which the present invention pertains, and the present invention will only be defined by the scope of the claims.

It is also to be understood that the terms used in this specification are for the purpose of describing embodiments only and is not intended to limit the present invention. In this specification, the singular form also includes the plural form, unless the context indicates otherwise. It is to be understood that the terms “comprise” and/or “comprising” used in the specification do not exclude the presence or addition of one or more other components other than stated components. Like reference numerals refer to like components throughout the specification and “and/or” includes respective mentioned components and all one or more combinations of the components. Although the terms “first”, “second”, and the like are used for describing various components, these components are not confined by these terms. These terms are merely used for distinguishing one component from another component. Therefore, a first component to be mentioned below may be a second component in a technical concept of the present invention.

Unless otherwise defined, all terms (including technical and scientific terms) used in the present specification may be used as the meaning which may be commonly understood by the person with ordinary skill in the art, to which the present invention pertains. Further, terms defined in commonly used dictionaries should not be interpreted in an idealized or excessive sense unless expressly and specifically defined.

Terms used in this specification are described.

An object represents a group of elements for representing a structural formula. For example, the structural formula may consist of characters, numbers, symbols, and figures. Specifically, the characters may represent atoms or compounds constituting the structural formula, the numbers may represent the number of atoms or charges, the symbols may represent positive charges (+), negative charges (−), or brackets representing specific repeating compound groups, and single line segments, double line segments, triple line segments, dotted lines, and the like, termed figures here for convenience, may represent chemical bonds.

The structural formula in this specification may be understood as one graph. The graph is a data structure consisting of points and lines connecting the points. Here, the point is referred to as a node, and the line is referred to as an edge. In other words, the structural formula may be understood as one graph in which at least one atom or compound corresponding to the node is linked by at least one bond corresponding to the edge. The structural formula may also include a bracket pair. The brackets represent a group of atoms and bonds repeated in the structural formula.

Hereinafter, embodiments of the present invention will be described with reference to drawings.

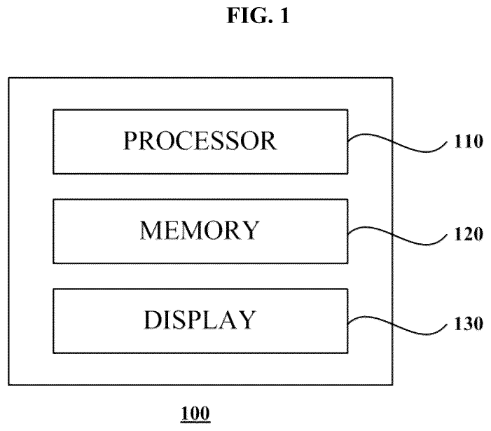

FIG. 1 is a block diagram of a device for training data generation according to an embodiment of the present invention.

The training data generation device 100 may include a processor 110, a memory 120, and a display 130. The processor 110, the memory 120, and the display 130 may be electrically connected to each other.

The processor 110 may load a first chemical formula data 200 including at least one packet of node information 210 and at least one packet of edge information 220 from the memory 120 or an external device. The processor 110 may also load the at least one feature variable 300 from the memory 120 or the external device.

The processor 110 may set, for the at least one loaded feature variable 300, a setpoint of each feature variable 300 to a predetermined first setpoint set 400.

The processor 110 may generate a structural formula in a form in which atoms or compounds are chemically bonded, based on at least one packet of node information and at least one packet of edge information being included in structure data. Therefore, the processor 110 may generate, based on first chemical formula data 200, a first structural formula by using an object set that is applied to the predetermined first setpoint set 400.

The processor 110 acquires, according to an input for changing the setpoint of the at least one feature variable 300, a second setpoint set 500 or a second setpoint set 600 in which the setpoint of a corresponding feature variable is changed.

An input for changing the setpoint of the feature variable 300 may be input from a user. In addition, the setpoint of the feature variable 300 may be randomly changed by the processor 110. In this case, the setpoint of each feature variable 300 may be randomly changed within a predetermined range or condition. The processor 110 may connect any structural formula to the first structural formula.

In this way, a large amount of structural formula training data having various shapes may be generated.

Further, the user's input and a processing of the processor 110 may be combined to change the setpoint of the feature variable 300. For example, when the user selects one specific variable among the feature variables 300, the processor 110 may randomly change the setpoint of the feature variable within a predetermined range. In addition, when the user inputs the number of structural formulas to be generated, the processor 110 may randomly change any feature variable so that the corresponding number of structural formulas is generated. In this way, a large amount of structural formula training data having shapes desired by the user may be generated.

The processor 110 may acquire the second setpoint set 500 or the second setpoint set 600 wherein, in either second setpoint set, a setpoint of at least one of the feature variables 300 is changed relative to its setpoint in the first setpoint set.

The processor 110 may generate a second structural formula using an object set that is applied to the second setpoint set 500 or the second setpoint set 600. The processor 110 may generate training data including the generated second structural formula.

The memory 120 may store at least one feature variable 300 for determining a feature of an object representing a structural formula, and a first setpoint set 400 predetermined for each of the at least one feature variable 300.

The display 130 may output the structural formula. Specifically, the display 130 may output the first structural formula generated by using the object set applied to the first setpoint set 400, and the display 130 may output the second structural formula generated by using the object set applied to a second setpoint set 500 or second setpoint set 600, where the second setpoint sets are changed relative to the first setpoint set.

The display 130 may output the first chemical formula data 200, the feature variable 300, the predetermined first setpoint set 400, and the changed second setpoint sets 500 and 600. The user may change the feature variable 300 of the desired object after confirming the first structural formula through the display 130, and confirm the changed second structural formula.

In addition, the display 130 may output the second structural formula separately for each time point at which the changed second setpoint set is acquired.

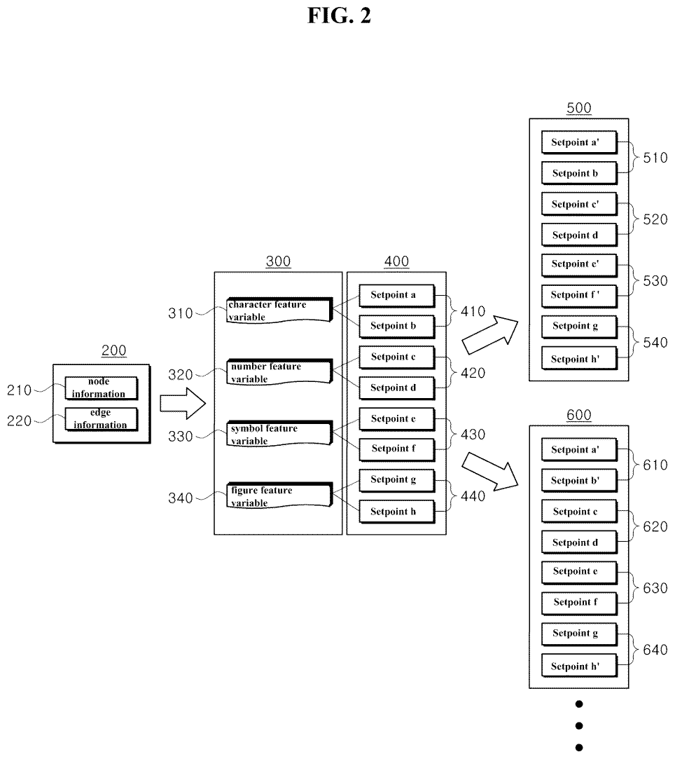

FIG. 2 is a diagram for describing a process of generating a structural formula based on chemical formula data according to an embodiment of the present invention.

FIG. 2 illustrates a process of generating various types of structural formulas based on the loaded first chemical formula data 200. Specifically, a process of setting a value of a specific variable 300 of an object for representing the first chemical formula data 200 as a structural formula to a predetermined first setpoint set 400, acquiring the second setpoint set 500 or the second setpoint set 600 according to an input of changing the setpoint of the specific variable 300, and generating the structural formula using the object set to the second setpoint set 500 or the second setpoint set 600 is illustrated.

The first chemical formula data 200 includes information about at least one node 210 and information about at least one edge 220. For example, the first chemical formula data 200 may include a list of atoms constituted by the structural formula, a multiplicity of atoms linked by chemical bonds and the bond order (single, double, or triple) of the chemical bonds, coordinates on a 2D or 3D space for each atom, the number of atoms, the number of chemical bonds, the total charge of the structural formula, a charge residing with each atom and attributes related to isomers, and the like. The first chemical formula data 200 may be in the format of an MOL file, a standard molecular data file.

The feature variable 300 may include a character feature variable 310, a number feature variable 320, a symbol feature variable 330, and a figure feature variable 340.

The character feature variable 310 may include a position of a character, a font type, an outline thickness of a character, a size of each character, a size by word, a spacing between characters, a color of a character, and an angle at which a character is tilted.

The number feature variable 320 may include a position of a number, a font type, an outline thickness of a number, a size of a number, a spacing between a number and a character, a spacing between numbers, a color of a number, and an angle at which a number is tilted.

The symbol feature variable 330 may include a position of a symbol, a font type, an outline thickness of a symbol, a size of a symbol, a spacing between a symbol and a character, a spacing between a symbol and a number, a spacing between symbols, a color of a symbol, and an angle at which a symbol is tilted.

The figure feature variable 340 may include a position of a figure, a type, an outline thickness of a figure, a size of a figure, a spacing between a figure and a character, a spacing between a figure and a number, a spacing between a figure and a symbol, a spacing between figures, a color of a figure, and an angle at which a figure is tilted.

By providing various feature variables 300 in this way, it is possible to generate a large amount of structural formulas from data describing one chemical formula. Meanwhile, it is understood that, in addition to the variables listed above, all variables that determine a feature of an object constituting the structural formula may be included in the feature variable 300.

The first setpoint set 400 is a group of predetermined setpoints. The first setpoint set 400 may include a setpoint 410 for a character feature variable 310, a setpoint 420 for a number feature variable 320, a setpoint 430 for a symbol feature variable 330, and a setpoint 440 for a figure feature variable 340. Although only eight setpoints of the setpoints a to h are illustrated in the drawings, it is understood that the setpoints are not limited thereto.

For example, the setpoint “a” may have a setting of “Arial” as a value for determining a font of a character, and the setpoint “b” may have a setting of “10” as a value of determining a size of the character.

The second setpoint set 500 is a set including setpoints in which the setpoints a, c, e, f, and h of the first setpoint set 400 are changed to setpoints a, c, e, f, and h′, respectively. The second setpoint set 600 is a set including setpoints in which the setpoints a, b, and h of the first setpoint set 400 are changed to setpoints a, b, and h′, respectively. The remaining setpoints which are not changed are the same as the setpoints of the first setpoint set 400.

One structural formula may be generated by using an object set applied to the second setpoint set 500, and another type of structural formula may also be generated by using the object set applied to the second setpoint set 600.

Although FIG. 2 illustrates a case where two second setpoint sets 500 and 600 are formed, it can be understood that numerous second setpoint sets may be formed according to a combination of changed setpoints.

Hereinafter, the present invention will be described using embodiments of specific structural formulas.

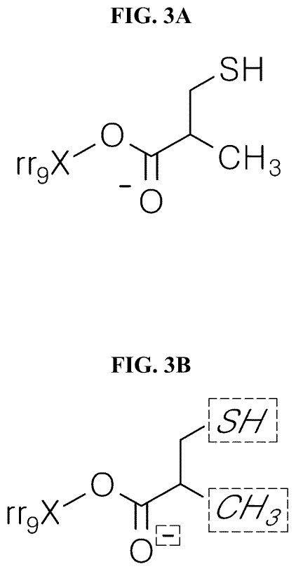

FIG. 3A illustrates a first structural formula generated by a predetermined setpoint, and FIG. 3B illustrates a second structural formula in which specific variable setpoints are changed for some characters, numbers, and symbols according to an embodiment of the present invention. Specifically, a portion indicated by a dotted line in FIG. 3B shows an object changed in comparison with FIG. 3A.

As described above, the processor 110 may load the first chemical formula data 200 including node information and edge information, and this may generate the structural formula of FIG. 3A, and the processor 110 may load the feature variable 300 in order to generate the first structural formula as illustrated in FIG. 3A by using the object set applied to the predetermined first setpoint set 400.

The processor 110 may acquire, according to an input for changing a setpoint of at least one feature variable 300, a second setpoint set 500 in which a setpoint of the feature variable is changed, and the processor 110 may then generate a second structure as illustrated in FIG. 3B by using an object set applied to the second setpoint set 500.

In FIG. 3A, SH represents a combination of characters, and CH₃ represents a combination of characters and a number. Accordingly, the processor 110 may match the character feature variable 310 to the SH, and match the character feature variable 310 and the number feature variable 320 to the CH₃, respectively. FIG. 3B illustrates a case where, for the SH, setpoints indicating the font and a tilting degree are changed within the character feature variable 310, and, for the CH₃, setpoints indicating the font and the tilting degree within the character feature variable 310 and the number feature variable 320 are changed.

Further, a charge (−) of O in FIG. 3A is a symbol. Therefore, the charge (−) of O may be matched to the symbolic feature variable 330. FIG. 3B illustrated that a setpoint indicating a position of the charge (−) of O is changed.

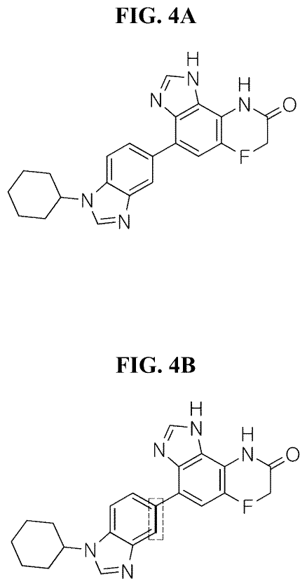

As illustrations of other embodiments, FIGS. 4 to 7 will be described.

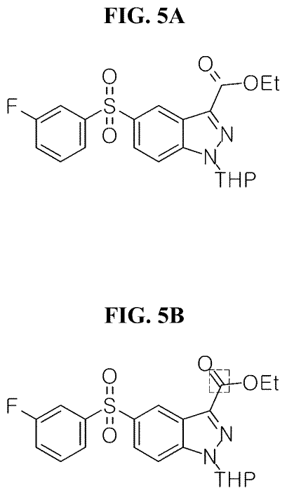

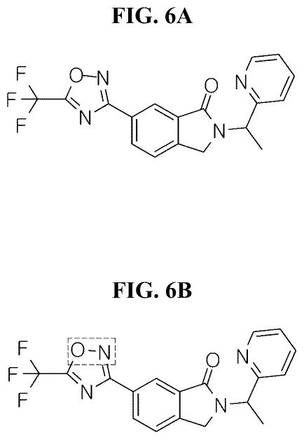

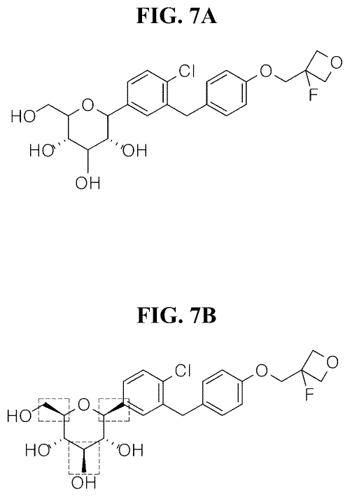

FIG. 4A, FIG. 5A, FIG. 6A, and FIG. 7A illustrate the first structural formula generated by the predetermined first setpoint set 400, and FIG. 4B, FIG. 5B, FIG. 6B, and FIG. 7B illustrate for each case the second structural formula for which setpoints of some figures (representing chemical bonds) are changed according to an embodiment of the present invention. Specifically, portions indicated by dotted lines in FIG. 4B, FIG. 5B, FIG. 6B, and FIG. 7B show objects changed in comparison with FIG. 4A, FIG. 5A, FIG. 6A, and FIG. 7A, respectively.

The combination of chemical bonds in each of the first structural formulas illustrated in FIG. 4A, FIG. 5A, FIG. 6A, and FIG. 7A is a figure object. Therefore, the processor 110 may match the figure feature variable 340 to each combination.

FIG. 4B illustrates a case where a setpoint indicating the thickness in the figure feature variable 340 is changed to increase with respect to a single combination (bond) of the dotted line indication portion as compared with FIG. 4A. FIG. 5B illustrates a case where a setpoint indicating a tilting angle of a figure and a spacing between the figures in the figure feature variable 340 are changed with respect to a double bond of the dotted line indication portion as compared with FIG. 5A. FIG. 6B illustrates a case where a setpoint indicating a length in the figure feature variable 340 is changed to decrease with respect to a single bond of the dotted line indication portion as compared with FIG. 6A. FIG. 7B illustrates a case where a setpoint indicating a type in the figure feature variable 340 is changed from a line segment to a wedge with respect to the single bond of the dotted line indication portions as compared with FIG. 7A.

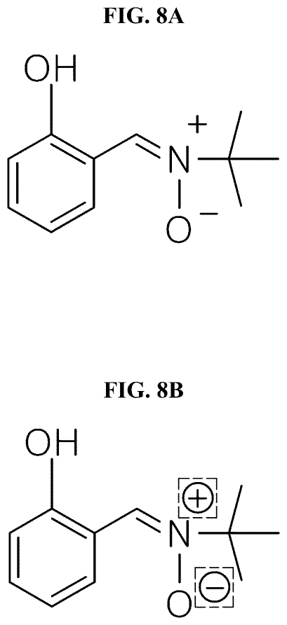

As another embodiment, FIGS. 8A and 8B will be described.

FIG. 8A illustrates a first structural formula generated by a predetermined first setpoint set 400, and FIG. 8B illustrates a second structural formula in which setpoints of some figures (chemical bonds) are changed with respect to the structural formula of FIG. 8A according to an embodiment of the present invention. Specifically, a portion indicated by a dotted line in FIG. 8B shows an object changed in comparison with FIG. 8A.

In FIG. 8A, charges (+ and −) of each atom represent a symbol object. Therefore, the processor 110 may match the symbol feature variable 330 to each symbol object. By changing a value of the matched symbol feature variable 330, a simple symbol (+, −) indicating charge as illustrated in FIG. 8A may be changed to a symbol illustrated in FIG. 8B. In addition, the positions of the symbols (+ and −) may also be changed as described above in the description of FIGS. 3A and 3B.

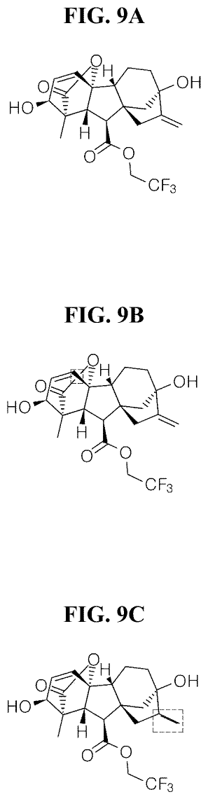

FIGS. 9A to 9C illustrate a case where two second structural formulas are generated from one first structural formula. FIG. 9B illustrates a case where the setpoint indicating the spacing between figures (portions of the same C—O single bond) in the figure feature variable 340 is changed to increase with respect to a perceived intersection between two single noncoincident bonds in the dotted line indicated portion of the two dimensional representation as compared with FIG. 9A. FIG. 9C illustrates a case where a setpoint indicating a spacing between line segments in the figure feature variable 340 is changed to decrease with respect to a double bond of the dotted line indication portion as compared with FIG. 9A, where the two line segments together represent a double bond.

As described above, various types of structural formulas may be generated in large quantities by changing predetermined setpoints for the feature variables 300 of characters, numbers, symbols, and figures that are objects constituting the structural formula.

In the second structure formula generated by the processor 110, a setpoint is set for each of feature variables 300 of objects constituting the second structural formula, and thus, the second structural formula may be utilized to annotate each object with the setpoint of the feature variable 300. Since each object is annotated, an artificial intelligence model that uses the annotated object as training data may improve a performance thereof.

In general, data annotation can be performed by performing bounding box processing of an object included in an image, and inputting attribute information of the object subjected to the bounding box processing. Such an annotation is also referred to as data labeling. Then, a data set corresponding to a task result of the annotation can be calculated in the form of a Java script object notation (JSON) file.

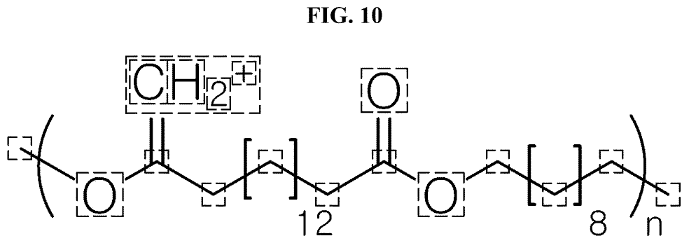

FIG. 10 illustrates a structural formula annotated to an object that constitutes a node of the second structural formula.

Dotted line portions shown in FIG. 10 encompass objects constituting the node. The feature variable 300 is matched to each of the objects, and a specific setpoint is set for each feature variable 300. For example, for O, C, and H, a setpoint of the character feature variable 310 may be set, for a number 2, a setpoint of the number feature variable 320 may be set, and for a node without a character and a number, a setpoint of the figure feature variable 340 may be set. As described above, the object constituting the node of the second structural formula may be formed by being annotated with a setpoint set for the feature variable 300 of the object.

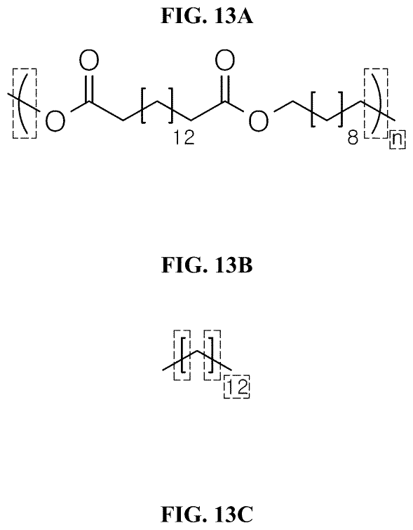

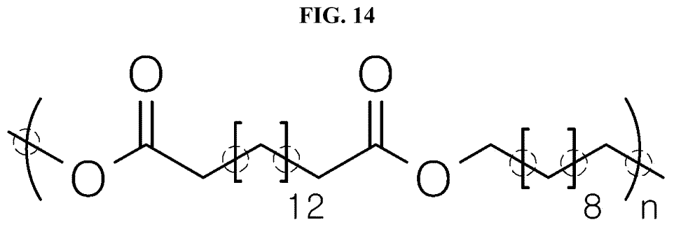

FIG. 11, FIG. 13A, and FIG. 14 illustrate the same second structural formula.

FIG. 11 illustrates a structural formula annotated to an object that constitutes an edge of the second structural formula. FIGS. 13A-13C illustrate a structural formula in which objects constituting a bracket of the second structural formula are annotated and FIG. 14 illustrates a structural formula in which a point where the object constituting the bracket of the second structural formula intersects with another object is annotated.

Dotted line portions shown in FIG. 11 encompass objects constituting the edge. For example, for single line segments and double line segments, a setpoint of the figure feature variable 340 may be set.

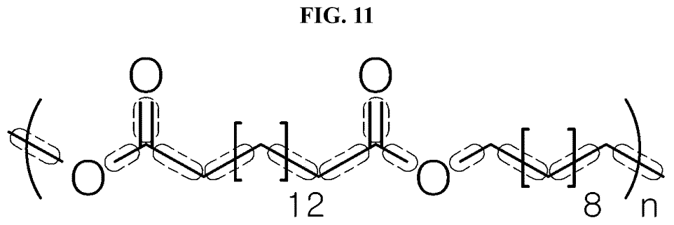

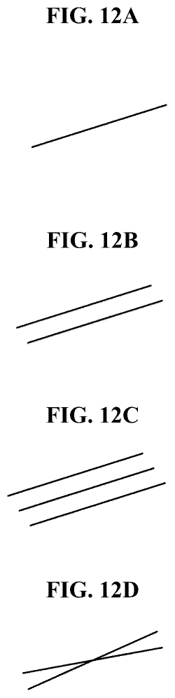

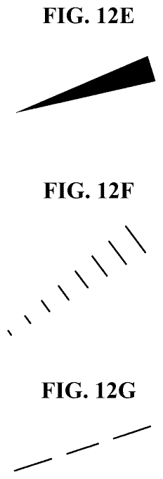

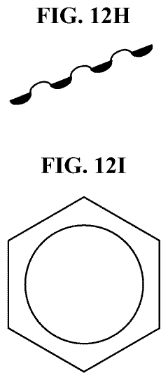

FIGS. 12A-12I illustrate types of objects constituting the edge according to embodiments of the present invention. FIG. 12A illustrates a single bond, FIG. 12B illustrates a double bond, FIG. 12C illustrates a triple bond, FIG. 12D illustrates a form of a cross-linked bond, FIGS. 12E and 12F illustrate a form of a bond for three-dimensionally expressing a bond, FIG. 12G illustrates a bond indicated by a dotted line, FIG. 12H illustrates a wavy bond, and FIG. 12I illustrates a bond of a resonance structure.

As described above, the object constituting the edge of the second structural formula may be formed by being annotated with a setpoint set for the feature variable 300 of the object.

Dotted line portions shown in FIGS. 13A-13C encompass objects constituting brackets.

The bracket pair of FIG. 13A shows a structure in which an entire structural formula included in the corresponding bracket is linked while being repeated n times.

FIG. 13B illustrates a part of FIG. 13A. The bracket of FIG. 13B shows that the structural formula included in the bracket is linked while being repeated 12 times.

FIG. 13C illustrates a part of FIG. 13A. The bracket of FIG. 13C shows that the structural formula included in the bracket is linked while being repeated 8 times.

As described above, the object constituting the bracket pair of the second structural formula may be formed by being annotated with a setpoint set for the feature variable 300 of the object.

A dotted line portion shown in FIG. 14 indicates a point where the object constituting the bracket and the object constituting the edge intersect with each other in the two dimensional structure representation. The intersection of the bracket and the edge may be annotated as illustrated in FIG. 14, separately from the intersection between the edge and the edge in FIG. 12D.

As described above, the object constituting the bracket and the object constituting the edge in the second structural formula may be formed by annotating a point where the object constituting the bracket and the object constituting an edge intersect with each other.

Such an annotation task may be automatically performed by the processor 110 of the training data generation device 100.

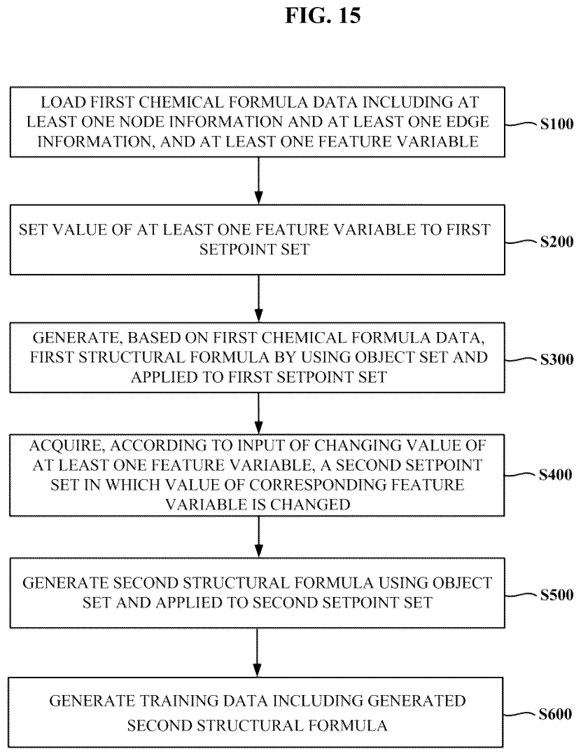

FIG. 15 is a block diagram for describing a method for generating training data according to an embodiment of the present invention.

The training data generation device 100 may load first chemical formula data 200 including at least one node information and at least one edge information, and at least one feature variable 300 (S100).

The training data generation device 100 may set a setpoint of the at least one feature variable 300 as a first setpoint set 400 (S200).

The training data generation device 100 may generate, based on the first chemical formula data 200, a first structural formula by using an object set and applied to the first setpoint set 400 (S300).

The training data generation device 100 can acquire, according to an input of changing the setpoint of the at least one feature variable 300, a second setpoint set 500 in which the setpoint of the corresponding feature variable is changed (S400).

The training data generation device 100 may generate a second structural formula by using an object set applied to the second setpoint set 500 (S500).

The training data generation device 100 may generate training data including the generated second structural formula (S600). In the second structural formula generated as described above, a setpoint set for each object may be annotated.

The memory may include at least one type of storage medium selected from a flash memory type storage medium, a hard disk type storage medium, a solid state disk (SSD) type storage medium, a silicon disk drive (SDD) type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk.

The disclosed embodiments may be implemented in the form of a recording medium storing instructions executable by the computer. The instructions may be stored in the form of a program code and when the instructions are executed by a processor, the instructions generate a program module to perform operations of the disclosed embodiments. The recording medium may be implemented as a computer readable recording medium.

The computer readable recording medium can include any of all kinds of recording media storing instructions which may be deciphered by the computer. For example, the recording media may include a read only memory (ROM), a random access memory (RAM), a magnetic tape, a magnetic disk, a flash memory, an optical data storage device, etc.

As described above, the disclosed exemplary embodiments are described with reference to the accompanying drawings. Those skilled in the art to which the present invention pertains will understand that the present invention may be implemented in a different form from that of the disclosed embodiments without changing the technical spirit or essential features of the present invention. The disclosed embodiments are exemplary and should not be construed as being limited.

The present invention relates to a training data generation device that machine-learns structural formula training data, and an artificial intelligence model trained by the training data generation device, and thus the present invention has an industrial applicability.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

1. A device for training data generation, the device comprising:
a processor; and
at least one memory electrically connected to the processor,
wherein the at least one memory stores at least one feature variable for determining a feature of an object representing a structural formula, and a first setpoint set predetermined for each of the at least one feature variable(s), and
wherein the processor
loads first chemical formula data including information about at least one node and information about at least one edge, and the at least one feature variable,
sets a setpoint of each of the at least one feature variable(s) to the corresponding first setpoint set,
generates, based on the first chemical formula data, a first structural formula by using an object set that is applied to the first setpoint set for each of the at least one feature variable(s),
acquires, according to an input of changing the setpoint of each of the at least one feature variable(s), a second setpoint set in which the setpoint of each corresponding feature variable is changed,
generates a second structural formula using an object set that is applied to the second setpoint set, and
generates training data including the generated second structural formula.

2. The device of claim 1, wherein a new object constituting a node of the second structural formula is formed by being annotated with the setpoint set for the feature variable of the corresponding existing object.

3. The device of claim 1, wherein a new object constituting an edge of the second structural formula is formed by being annotated with the setpoint set for the feature variable of the corresponding existing object.

4. The device of claim 3, wherein a new object constituting a bracket of the second structural formula is formed by being annotated with the setpoint set for the feature variable of the corresponding existing object.

5. The device of claim 4, wherein the object constituting the bracket and the object constituting the edge in the second structural formula are formed by annotating a point where the object constituting the bracket and the object constituting the edge intersect with each other.

6. The device of claim 1, wherein the training data generation device includes a display outputting a structural formula, wherein the processor acquires a second setpoint set at each of a plurality of time points, and
wherein the display outputs the first structural formula and the second structural formula, and outputs the second structural formula separately for each time point at which the changed second setpoint set is acquired.

7. The device of claim 1, wherein each of the at least one feature variable(s) includes at least one of a type, a thickness, a length, a size, and a color of the object, a spacing between objects, and an angle at which the object is tilted.

8. The device of claim 1, wherein the training data is used for training an artificial intelligence model for predicting chemical formula data including information about at least one node and information about at least one edge.

9. A method for training data generation, which method is performed by a training data generation device, the method comprising:
storing, by the training data generation device, at least one feature variable for determining a feature of an object representing a structural formula, and a first setpoint set predetermined for each of the at least one feature variable(s);
loading first chemical formula data including information about at least one node and information about at least one edge, and the at least one feature variable(s);
setting a setpoint of each of the at least one feature variable(s) to the respective first setpoint set;
generating, based on the first chemical formula data, a first structural formula by using a first object set and applying the first object set to the first setpoint set for each feature variable;
acquiring, for each at least one feature variable, according to an input of changing the setpoint(s) of the at least one feature variable(s), a second setpoint set in which the setpoint of the corresponding feature variable is changed relative to the first setpoint set;
generating a second structural formula using a second object set that is applied to the second setpoint set; and
generating training data including the generated second structural formula.

10. The method of claim 9, wherein an object constituting a node of the second structural formula is formed by being annotated with the second setpoint set(s) for the feature variable(s) of the corresponding object of the second object set.

11. The method of claim 9, wherein an object constituting an edge of the second structural formula is formed by being annotated with the second setpoint set(s) for the feature variable(s) of the corresponding object of the second object set.

12. The method of claim 9, wherein an object constituting a bracket of the second structural formula is formed by being annotated with the second setpoint set(s) for the feature variable(s) of the corresponding object of the second object set.

13. The method of claim 9, wherein an object constituting a bracket and an object constituting an edge in the second structural formula are formed by annotating a point where the object constituting the bracket and the object constituting the edge intersect with each other.

14. The method of claim 9, wherein the at least one feature variable includes at least one of a type, a thickness, a length, a size, and a color of the object, a spacing between objects, and an angle at which the object is tilted.

15. A computer program stored in a computer-readable recording medium, coupled with a device that is hardware, to execute the method for generating training data of claim 9.
